(19) United States
(12) Patent Application Publication
(10) Pub. No.: US 2025/0017995 A1
(43) Pub. Date: Jan. 16, 2025

(54) MAMAKU EXTRACT FOR TREATING ECZEMA

(71) Applicant: ORA INNOVATION NEW ZEALAND LIMITED, Auckland (NZ)

(72) Inventors: Tessa Rau-te-kura DAVIS, Hamilton (NZ); David Paul HARRIS, Hamilton (NZ)

(73) Assignee: ORA INNOVATION NEW ZEALAND LIMITED, Auckland (NZ)

(21) Appl. No.: 18/711,178

(22) PCT Filed: Nov. 18, 2022
(86) PCT No.: PCT/NZ2022/050146

§ 371 Date: May 17, 2024

(30) Foreign Application Priority Data
Nov. 19, 2021 (NZ) 782513

(51) Int. Cl. A61K 36/12; A61K 9/00; A61K 47/10; A61K 47/22; A61K 47/36; A61P 17/00; B01D 11/02

(52) U.S. Cl. CPC A61K 36/12; A61K 9/0014; A61K 47/10; A61K 47/22; A61K 47/36; A61P 17/00; B01D 11/0288; A61K 2236/15; A61K 2236/331

(57) ABSTRACT

An extract from plant material of the New Zealand native fern tree mamaku (Cyathea medullaris) which has been processed under gentle conditions to provide an effective treatment for eczema.

### TECHNICAL FIELD

The invention relates to an extract from the New Zealand native fern tree Cyathea medullaris, known as the black free fern or by its Mjori name mamaku. In particular, the invention relates to an extract from mamaku plant material which has been processed under controlled conditions to provide an effective treatment for eczema.

### BACKGROUND OF THE INVENTION

Mamaku (Cyathea medullaris, also known as Sphaeropteris medullaris) is the Maori name for the black tree fern which is native to New Zealand. Other names for mamaku include korau, Alsophila extensa, Cyathea polyneuron, and Polypodium medullare. Mamaku is the tallest and fastest growing New Zealand native fern, and can grow to a height of about metres above ground.

Mamaku has been used in traditional Maori herbal medicine for skin conditions and for gut health. The slimy flesh and the mucilage obtained directly from a cut mamaku frond and applied to the skin has been used for treating various wounds and skin conditions such as cuts and sores, poisoned hands, swollen feet, inflamed breasts, chafing, dryness and sunburn.

Extracts of mamaku plant material have also been used for improving skin appearance. For example, WO 2013/022788 describes compositions for skin care products derived from various plant ingredients one of which is an extract prepared from Cyathea medullaris. The compositions are said to improve skin elasticity, skin thickness and skin hydration, and avoid age-related skin features such as wrinkles.

Extracts of mamaku have been found to have distinctive rheological properties due to the presence of glucuronomannan polysaccharides. See Goh et al., Biomacromolecules, 2007, 8, 3414-3421; Matia-Merino et al., Carbohydrate Polymers, 2012, 87, 131-138; and Wee et al., International Journal of Biological Macromolecules, 2014, 70, 86-91. Mamaku extracts were found to consist of large polysaccharides of molecular weight approximately 2,000,000 Daltons. It is thought that the polysaccharide component is responsible for the unique rheological properties of mamaku extracts including high viscosity. It is also likely that the polysaccharide component gives mamaku extracts their skin barrier function which helps to prevent transepidermal water loss (TEWL) when applied to the skin. See de Camargo et al., Brazilian Journal of Pharmaceutical Sciences, 2012, 48:3, 547-5552012; and Lautenschlager, Kosmetische Praxis, 2009, 4, 12-152009. The long chains of polysaccharide molecules are thought to form a network thereby creating a thin film on the surface of the skin.

Eczema, also known as atopic dermatitis, is the most common inflammatory skin disease worldwide and presents as generalised skin dryness, itchiness and rash. Eczema results from a complex interplay between environmental and genetic factors, for which there is no known cure. However, eczema can be controlled with various treatments, including topical treatments in the form of lotions, gels, foam, creams and ointments. Such topical treatments include emollients (which moisturise the skin), antiseptics (which prevent or treat infections), steroids (which are anti-inflammatory, immunosuppresive, anti-proliferative or vasoconstrictive), calcineurin inhibitors (which are immunomodulators), and the use of phototherapy (electromagnetic radiation).

Some eczema treatments have known side effects. For example, the side effects of steroid medications include skin thinning, stretch marks, easy bruising, tearing of the skin, enlarged blood vessels and localised increased hair thickness and length. Side effects of calcineurin inhibitors include potential damage to the kidneys.

Some eczema treatment formulations have other negative attributes. For example, greasy creams containing emollients can make the skin uncomfortable under clothing and can stain clothing. Paraffin-based creams can soak into clothing causing a fire hazard. Greasy emollients can make the skin slippery which is a danger when bathing or when holding or grasping objects. Surfactants, such as sodium lauryl sulphate, which are necessary to mix oil and water-based treatments, can irritate the skin thereby exacerbating the eczema. Some treatments, such as coal tar, have an undesirable smell.

Although WO 2013/022788, referred to above, mentions eczema in a list of a skin-related conditions, there is no description provided of any composition properties that suggest efficacy for the treatment of eczema. Notably, the mamaku extract described in WO 2013/022788 is in the form of a powder, indicating significant and aggressive processing of extract material which likely damages polysaccharide compounds and other compounds in the extract.

A product known as Vital Essence™ containing a mamaku extract from New Zealand was developed by Lucas Meyer Cosmetics in 2002. It is said that this extract showed interesting anti-aging properties, and that the extract makes it possible to intensify the cytoskeleton, to stimulate cell proliferation, and to increase the synthesis of fibroblasts, proteoglycans and glycosaminoglycans. Lucas Meyer launched a new product in 2002 under the name Liftessence™ prepared from a fern extract of the genus Cyathea. The product was marketed as improving the tone and firmness of the skin and reducing skin wrinkles.

The nature of the processes used for obtaining extracts from plant material and for the subsequent processing of extracts into products for use, can have a dramatic effect on their biological, chemical and physical properties. Known extraction and processing procedures utilise potentially harsh chemical, mechanical or physical conditions such as washing, cutting, pulverising, pressing, drying, grinding, water extraction, organic solvent extraction, liquid-liquid partitioning, ultrasound treatment, microwaving, steeping, macerating, coarse filtration, fine filtration, ultra-filtration, heating, reduced pressure, refluxing, activated carbon, chromatography, acid/alkali treatment, mixing, rotor/stator homogenising, high pressure homogenising, centrifugation, decanting, sieving, freeze/thawing, detergents, and in some cases the addition of chemicals such as EDTA (chelating), detergents, acids or alkalis, and dispersants.

FR2945446 describes mixing a composition with an extract of Cyathea medullaris for increasing the viscosity or modifying the behaviour of a composition. The patent describes the preparation of the extract by harsh or high energy methods know in the art, i.e. typically by crushing Cyathea leaves and macerating them in water, followed by centrifugation, ultrafiltration, alcohol precipitation, activated carbon bleaching, clarification or filtration, then drying by lyophilisation or atomisation.

The applicant has surprisingly found that an extract obtained from Cyathea medullaris has a different physicochemical profile and is beneficial for the treatment of eczema when prepared under conditions that are gentle and not aggressive when compared to the processing conditions employed for known Cyathea medullaris extracts.

It is therefore an object of the invention to provide a composition comprising an extract from Cyathea medullaris useful for the treatment of eczema, or to at least provide an alternative to known therapeutic treatments.

### SUMMARY OF THE INVENTION

In one aspect the invention provides an aqueous extract of the black tree fern Cyathea medullaris wherein the extract is prepared by:
(i) cutting fronds of Cyathea medullaris to give plant material having an average portion size of 0.1 to 10 cm;
(ii) heating a mixture of the plant material of step (i) and water at a temperature of 45 to 60° C. with laminar mixing or transitional flow mixing; and
(iii) removing solid material from the mixture to give the aqueous extract.

In some embodiments of the invention the amount of plant material in water in step (ii) is 20-30% by weight.

In some embodiments of the invention the plant material is heated in step (ii) for 30 minutes to 2 hours.

In some embodiments of the invention the average portion size of the plant material is 0.2 to 5 cm.

In some embodiments of the invention the temperature of the water in step (ii) is 50-55° C.

In some embodiments of the invention removing the solid material in step (iii) is by filtration under laminar conditions, for example under low pressure or under gravity.

In some embodiments of the invention the extract comprises glucuronomannan polysaccharides in the amount of 0.05 to 0.1% by weight of the extract.

In some embodiments of the invention the extract additionally comprises glycerin and/or xanthan gum.

In some embodiments of the invention the plant material comprises fronds of the Cyathea medullaris plant.

In a second aspect the invention provides a topical composition for use in treating eczema comprising the extract of the invention.

In some embodiments of the invention the composition further comprises one or more preservatives, such as dehydroacetic acid and benzyl alcohol.

In some embodiments of the invention the composition further comprises xanthan gum and/or glycerin.

In a second aspect the invention provides the use of an aqueous extract of the black tree fern Cyathea medullaris for the treatment of eczema wherein the extract is prepared by:
(i) cutting fronds of Cyathea medullaris to give plant material having an average portion size of 0.1 to 10 cm;
(ii) heating a mixture of the plant material of step (i) and water at a temperature of 45 to 60° C. with laminar mixing or transitional flow mixing; and
(iii) removing solid material from the mixture to give the aqueous extract.

In another aspect the invention provides a method of treating eczema comprising administering to a subject an aqueous extract the black tree fern Cyathea medullaris wherein the extract is prepared by:
(i) cutting fronds of Cyathea medullaris to give plant material having an average portion size of 0.1 to 10 cm;
(ii) heating a mixture of the plant material of step (i) and water at a temperature of 45 to 60° C. with laminar mixing or transitional flow mixing; and
(iii) removing solid material from the mixture to give the aqueous extract.

In another aspect the invention provides a process for preparing an aqueous extract of the black tree fern Cyathea medullaris comprising:
(i) cutting fronds of Cyathea medullaris to give plant material having an average portion size of 0.1 to 10 cm;
(ii) heating a mixture of the plant material of step (i) and water at a temperature of 45 to 60° C. with laminar mixing or transitional flow mixing; and
(iii) removing solid material from the mixture to give the aqueous extract.

### BRIEF DESCRIPTION OF THE FIGURES

FIG. 1 shows the results of skin hydration experiments using an extract from mamaku plant material.

FIG. 2 shows the results of transepidermal water loss experiments using an extract from mamaku plant material.

FIG. 3 shows the results of a spreadability test for mock cosmetic products compared with commercially available Ora Hydragel.

FIG. 4 shows the results of a viscosity test for mock cosmetic products compared with commercially available Ora Hydragel.

FIG. 5 shows the results of a hydration test for mock cosmetic products compared with commercially available Ora Hydragel.

FIG. 6 shows the results of a TEWL test for mock cosmetic products compared with commercially available Ora Hydragel.

### DETAILED DESCRIPTION

The term “mamaku” means the New Zealand native black tree fern which is also known as korau, Cyathea medullaris, Sphaeropteris medullaris, Alsophila extensa, Cyathea polyneuron, and Polypodium medullare.

The term “frond” means the part of the plant that includes the stipe and rachis but excludes the pinna (leaves).

The term “laminar mixing” means mixing of a fluid system at a Reynolds number less than 2,000.

The term “transitional flow mixing” means mixing of a fluid system at a Reynolds number greater than 2,000 and less than 4,000.

The term “turbulent mixing” means mixing of a fluid system at a Reynolds number greater than 4,000.

The term “Reynolds number” or “Re” means the number describing the boundary point between laminar and turbulent flow.

The term “eczema” means any superficial inflammatory process involving primarily the epidermis marked by redness, itching, minute papules and vesicles, weeping, oozing and crusting, and later by scaling, lichenification and often pigmentation. Eczema is also known as atopic dermatitis.

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the invention, various assays, methods, devices and materials are now described.

It is intended that reference to a range of numbers disclosed herein (for example, 1 to 10) also incorporates reference to all related numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used in this specification, the words “comprises”, “comprising”, and similar words, are not to be interpreted in an exclusive or exhaustive sense. In other words, they are intended to mean “including, but not limited to”.

Those skilled in the art will appreciate that the invention described in this specification is susceptible to variations and modifications other than those specifically described. It is to be understood that the invention includes all such variations and modifications. The invention also includes all of the steps or features referred to or indicated in this specification, individually or collectively, and any and all combinations or any two or more of said steps or features.

The invention is not to be limited in scope by the specific examples described in this specification, which are intended for the purpose of exemplification only. Functionally equivalent products, compositions and methods are clearly within the scope of the invention.

Any reference to prior art documents in this specification is not to be considered an admission that such prior art is widely known or forms part of the common general knowledge in the field.

The invention is based on the applicant's finding that the use of gentle non-aggressive conditions for obtaining and processing an extract from mamaku fronds has a beneficial effect on the resulting product in terms of its physicochemical profile. The applicant found that this product is effective for the treatment of eczema compared to products prepared from a mamaku extract under traditional more aggressive and high energy processing conditions, such as high temperature, high mechanical shear stresses, and very high pressures or very low pressures.

Mamaku extracts prepared by Goh (Biomacromolecules, 2007, 8, 3414-3421) and Wee (International Journal of Biological Macromolecules, 2014, 70, 86-91) were found to consist of large polysaccharides of molecular weight approximately 2,000,000. The principle polysaccharides found were glucuronomannans having a chain length of 1.9×10⁶, with branches of xylopyranosyl, galactopyranosyl, non-methylesterified glucopyranosyl uronic acid, and glucopyranosyl uronic acid. Other carbohydrates typically found in mamaku extracts include glucuronic acid, galactose, mannose, xylose, arabinose, galacturonic acid, rhamnose, fucose and glucose. Extracts also typically contain starch, minerals, protein, tannins, fats and fibre.

It is thought that the polysaccharide component is responsible for the unique rheological properties of mamaku extracts, including high viscosity. It is likely that the polysaccharide component gives mamaku extracts their skin barrier function, which helps to prevent transepidermal water loss when applied to the skin. The high molecular weight and long chains of polysaccharide likely form a network thereby creating a thin film on the surface of the skin.

Notably, the preparation method used by Goh involved blending cut pieces of mamaku fronds in a wet disintegrator and centrifugation of the crude extract which are high energy aggressive processing steps. It is known that certain types of processing can disrupt polysaccharide chains creating smaller molecules that display a reduced viscosity (Antilla et al. (2004) Agricultural and Food Science, 13, 80-87; Tosh et al. (2010) Journal of Agricultural and Food Chemistry, 58, 7723-7730). The applicant has deliberately avoided strong or high-energy processing conditions when extracting from raw mamaku plant material in order to maintain the integrity of compounds present in the plant material. The applicant found that this was important for creating a strong network and barrier on the skin.

Mamaku extracts have complex viscoelastic properties consisting of shear-thinning, shear-thickening, thixotropic, antithixotropic and Newtonian properties, depending on concentration, temperature and applied shear. They also exhibit rod climbing and self-siphoning properties.

The applicant has determined that a composition containing mamaku extract prepared according to the invention:
increases skin hydration in human volunteers
decreases transepidermal water loss (TEWL) in human volunteers
does not cause skin sensitivity in human volunteers subjected to repeated use.

The extract is an effective, natural, plant-based product for relieving the signs of eczema. It is not greasy, does not have any undesirable smell and does not contain irritants. The product is water-based, which is easily and quickly absorbed into the skin. This makes it desirable to use, especially for young children.

The process of the invention utilises gentle processing conditions. Firstly, the extraction step is carried out using water only as the solvent. In contrast to many known plant extraction procedures, no ethanol or any other water-miscible organic solvent is used in the extraction step.

Importantly, the temperature of the extraction step is minimised to avoid unwanted modification of chemicals present in the plant material, particularly long chain polysaccharides. The extract is maintained at a temperature to avoid the extract becoming too viscous and therefore difficult to handle. The applicant found that a suitable temperature is in the range 45 to 60° C., preferably about 50° C.

Rather than grind or pulverise the plant material in readiness for extraction, or any other form of harsh or aggressive treatment, the mamaku fronds are simply cut into pieces of about 0.1 to 20 cm in length, typically 10 to 20 cm in length, and then chopped to reduce the size of pieces to about 5 to 10 mm. This again avoids unwanted modification of compounds present in the plant material and maintains their integrity.

Once the plant material has been stirred in water at the desired temperature for a time sufficient to enable the extraction, usually around 0.5 to 2 hours, the solid material is separated from the aqueous extract. Again, this step is carried out under gentle conditions, for example by simple filtration under gravity. Typical known filtration procedures involve pumping and sieving using mechanical force under low shear rates.

Sufferers of eczema have inherently dry skin and are susceptible to a weaker skin barrier function. Furthermore, dry skin causes itchiness, leading to scratching, which further exacerbates the severity of the eczema. It is therefore desirable that topical treatments for eczema should help hydrate the skin, prevent further water loss through evaporation, and should not irritate the skin or cause undue itchiness.

The Examples described below further illustrate the invention.

Example 1 describes an extraction process of the invention. Mamaku fronds were cut into pieces and then chopped to reduce their size to about the size of a pea, e.g. 5-10 mm. Water was added and the mixture stirred gently at around 50° C. for about an hour. Gentle stirring is regarded as laminar mixing (Re<2,000) or transitional flow mixing (Re 2,000-4,000). Following filtration to remove solid material, the crude extract was freeze dried to give a soft hygroscopic solid, and then redissolved by mixing with water (Formulation 1). Preservatives (dehydroacetic acid and benzyl alcohol) were added to some of the crude extract, followed by xanthan gum and glycerin to give Formulation 2. Formulation 3 is aloe vera.

Example 2 is the procedure adopted for a trial investigating skin hydration, transepidermal water loss (TEWL), and skin irritation. The trial involved 17 human volunteers.

Example 3 describes an experiment showing the change in skin hydration, relative to initial baseline levels, for the test sites using Formulations 1, 2 and 3. Following treatment with sodium lauryl sulphate (SLS), all skin sites had significantly (p<0.01) reduced hydration.

All three formulations returned the skin hydration to baseline levels, or better, by day 3. The SLS control site did not return to baseline hydration levels until day 5. Both Formulations 2 and 3 significantly (p<0.01) hydrated the skin by day 3, with Formulation 2 restoring the skin hydration by up to 65% more hydrated compared with the undisrupted baseline, and continued to maintain high levels (>50%) over the period of the trial. This demonstrates a very fast healing response to SLS disruption. By comparison, Formulation 3 (aloe vera), was only able to achieve a maximum skin hydration level of 35%. Formulation 1 also gave a rapid initial healing response, returning the skin to baseline hydration levels but did not achieve any marked enhanced hydration levels. This could be attributed to the difficulty in applying this formulation, which does not contain any carrier to aid its application to the skin.

Example 4 describes an experiment showing the change in transepidermal water loss (TEWL) values, relative to initial baseline levels, for the test sites using Formulations 1, 2 and 3. Following treatment with SLS, all skin sites had significantly (p<0.01) increased TEWL values. An increase in TEWL values indicates an impairment in skin barrier function. A decrease represents an improvement. All three formulation treatments showed a decrease in TEWL levels during the period of the trial, whereas the SLS-treated site remained significantly (p>0.01) impaired compared to the control site. Formulations 2 and 3 both rapidly returned TEWL levels to within non-significant differences to the baseline levels by day 3. By comparison, Formulation 1 restored TEWL baseline levels by day 11. Formulation 2 continued to reduce the TEWL values such that by day 11 the TEWL values were significantly (p>0.01) lower than those of the undisrupted control site. This treatment therefore not only rapidly repaired the damage to the skin barrier function caused by the SLS disruption but also enhanced the barrier function of the skin.

Example 5 describes a clinical study to determine the level of skin irritation and skin sensitisation following repeated patch application. All subjects exhibited no dermal reactions throughout the course of the entire study and had scores of ‘0’. Based on the test population of 44 subjects with self-perceived sensitive skin and under the conditions of this study, the Formulation 2 product did not demonstrate any potential for eliciting dermal irritation or inducing sensitisation.

In a second trial, a crude extract was prepared according to Example 1 followed by the application of heat, ultrasound or mechanical agitation. Mamaku extract is characterised by its viscoelastic nature (Huang S., International Journal of Biological Macromolecules, 120, 1601-1609, 2018). When a mamaku extract is poured it has a self-siphoning quality (Goh et al., Biomacromolecules, 2007, 8, 3414-3421), and when it is stirred it exhibits rod climbing behaviour (Wee et al. International Journal of Biological Macromolecules, 2014, 70, 86-91). Untreated mamaku extract, made under the gentle conditions of Example 1, had all of these qualities.

Treating the extract with ultrasound or with high speed agitation dramatically reduced its viscosity and converted it to an easily pourable solution. Heat treatment, however, did not change its viscoelastic nature.

Mock cosmetic products were prepared from samples of these extracts according to Example 6 and their properties investigated. All four mock products (untreated, heat treated, ultrasound treated and agitation treated) were prepared from the same base formula of glycerol, xanthan gum and water. They all had good spreadability, similar to or slightly greater than that of the commercial mamaku extract cosmetic product Ora Hydrogel. See Example 7. The ability to apply and spread the products onto the skin, without any run-off or tackiness, was similar for each mock cosmetic product, and therefore any differences in the test results are due to the treatment that each extract received.

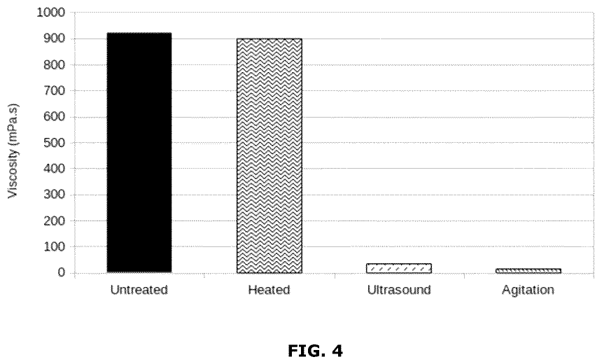

Example 8 describes an experiment investigating viscosity. FIG. 4 shows that the ultrasound-treated and the agitated extracts had a dramatically reduced viscosity, whereas the heat-treated extract had a viscosity similar to that of the untreated extract. These results are consistent with observations made by eye.

Example 9 describes the procedure for a second skin trial. The mock cosmetic products made with untreated, heat-treated, or ultrasound treated extract all provided significant hydration of the skin after days 3, 5 and 10 of the trial. These were significant at the P<0.01 level. There was no statistical significance between the samples, indicating that the type of extract treatment does not affect the ability of extract to hydrate the skin.

All mock cosmetic products showed reduced TEWL values and therefore a reduced loss of water from the surface of the skin, which is indicative of an increased barrier function. The heat-treated product was statistically significant at the p<0.05 or p<0.01 level on days 3 and 10. The untreated product was statistically significant at the p<0.05 level on day 3. Although the untreated product had similar TEWL values to the heat-treated product on day 10, it did not reach statistical significance. The untreated product gave an unusually low negative value on day 5 which is attributed to an anomalous result from a single participant. Omitting this anomalous result provides an average TEWL value of −7.4%, bringing it into line with the results for days 3 and 10. Ultrasound treatment was not significant on any of the days.

A topical formulation containing an extract of mamaku tree fern and other ingredients, when applied to the generalised skin at least twice daily, results in a reduction of the signs of eczema. See Example 10, and Rademaker M, Davis T, Harris P (2022), Australas J. Dermatol., 00, 1-2. A total of 67 participants applied an amount of Formulation 2 made according to Example 1 (containing 0.85% mamaku extract solids) twice daily over a two-week period. A total of 85% of the participants described an excellent/significant (27%), or good/moderate (58%), improvement of their skin, starting within two days of application of the Formulation. No patient had skin condition deterioration, and none reported treatment-emergent adverse effects. In the atopic eczema cohort (n=30), all participants commented on the cooling nature of the product, comparing it favourably to previous eczema treatments (e.g. topical corticosteroids and moisturisers), with no stinging or irritation. Most commented favourably that the gel was not greasy and was easy to apply. Erythema and itch improved in the majority of patients. Half of the patients using a topical corticosteroid reported decreased use, or discontinuation of, the corticosteroids (mostly 1% hydrocortisone). This open label, uncontrolled study indicates that a mamaku extract is effective for treating atopic eczema.

The invention is further described with reference to the following Examples. It will be appreciated that the invention as claimed is not intended to be limited in any way by these Examples.

### EXAMPLES

### Example 1: Extraction Process

Mamaku fronds (excluding the unfurled lamina) were cut into lengths of approximately 10-20 cm and chopped in a bowl chopper until pieces were approximately the size of a pea (e.g. 5-10 mm). The chopped plant material was then weighed and added to high quality water (reverse osmosis grade) at 50° C. so that the final concentration of mamaku was approximately 26% by weight. Alternatively, the plant material can be added to water at a temperature of 60° C., such that upon addition of the (colder) plant material to the hot water, the temperature of the mixture is immediately reduced to 50° C. The mixture was stirred gently at 50° C. for 1 hour.

The mixture was then transferred to food grade filter bags (mesh size approximately 60 to 100 μm). The hot liquid was separated from the plant pulp under gravity. The separated liquid extract was collected, weighed and freeze dried to give a soft hygroscopic solid. This extract is Formulation 1 in Examples 2-5.

Additionally, liquid extract was transferred to a kettle to be reheated to 50° C. with gentle stirring. Dehydroacetic acid and benzyl alcohol preservatives (Geoguard™) were added at a final concentration of 0.5 to 0.9% by weight and mixed for a few minutes until homogeneous. Xanthan gum (1.9% w/w) was added to modify the viscosity of the extract to form a gel. Finally, glycerin (10% w/w) was added as a humectant to facilitate absorption of the extract into the skin. This extract is Formulation 2 in Examples 2-5.

### Example 2: Trial 1 Procedure

The study comprised a total of 17 healthy participants, with a mean age of 37 and an age range of 26 to 56. Preference was given to those having a pre-existing skin condition which results in dry and irritated skin (e.g. eczema). Participants were instructed not to use any cosmetic products, aside from their standard soap/body wash, on their forearms for the duration of the study and for two weeks prior to the study.

Baseline measurements of the participants inner forearm skin were taken on day 1, and then the test sites were exposed to a 10% sodium lauryl sulphate (SLS) solution to elicit an irritant reaction of the skin. The SLS was applied to the skin using a 200 μL volume soaked into four layers of gauze in an 18 mm internal diameter Finn chamber, which was held in place on the test sites with kinesiology tape. After 2 hours, the Finn chambers were removed and the test areas were rinsed with water for 10 seconds and then patted dry. Skin measurements were retaken.

Participants were instructed to apply Formulations 1-3 to prescribed areas of the inner forearm skin, twice daily (morning and evening), to both arms commencing on the evening of day 1. To eliminate positional bias, these areas were randomly selected. Follow-up measurements were made on days 3, 5, 9 and 11.

Three formulations were tested:
Formulation 1: redissolved freeze dried extract prepared according to Example 1.
Formulation 2: liquid extract prepared according to Example 1 with added glycerin, xanthan gum and preservatives.
Formulation 3: commercially available aloe vera extract containing purified water, aloe barbadensis leaf extract, xanthan gum, glycerin, phenoxyethanol, and ethylhexylglycerin.

The initial baseline obtained on day 1, prior to exposure to SLS, was used to monitor the effect the irritant had on skin as well as to see if/when the skin returned to baseline, pre-SLS levels. The second baseline (SLS baseline), obtained two hours after exposure to SLS, was used to monitor the effect the different treatment options had on the recovery of the skin, after the skin irritation had been induced.

### Example 3: Skin Hydration

Measurement of skin hydration was based on a capacitance method which relies on the dielectric constant of water being much higher than other substances present in skin. The measuring capacitor shows changes to the capacitance according to the moisture content of the samples. A glass lamina separates gold tracks in the probe head from the skin to prevent current conduction in the sample. An electric field between the tracks generates charge separation, and a scatter field penetrates the first layer of skin during the measurement of the capacitance.

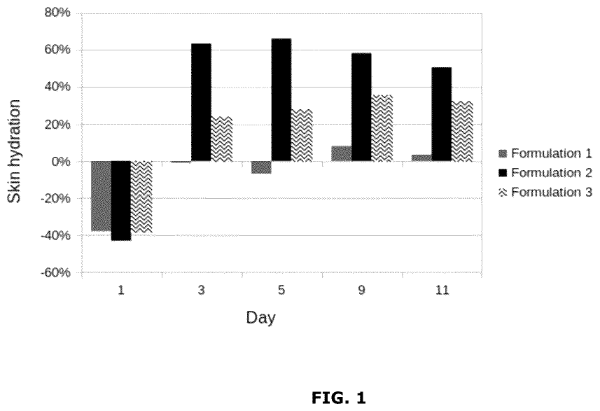

Measurements were taken at three different but nearby sites within the test area, with two readings taken at each site, giving a total of six measurements, according to EEMCO guidelines. The average of these six readings is shown in FIG. 1.

### Example 4: Transepidermal Water Loss (TEWL)

TEWL is the passive diffusion of water through the stratum corneum (SC) and is an indicator of the integrity of the barrier function of the SC. TEWL measurements are based on the estimation of the water vapour gradient in an open chamber. Fick's law of diffusion is the physical basis for the TEWL measurement. The diffusion flow indicates the mass per cm² being transported in a period. The measurements are made using two electrical probes housed inside a hollow tube of known diameter. The probes sense the moisture content and temperature adjacent to the skin.

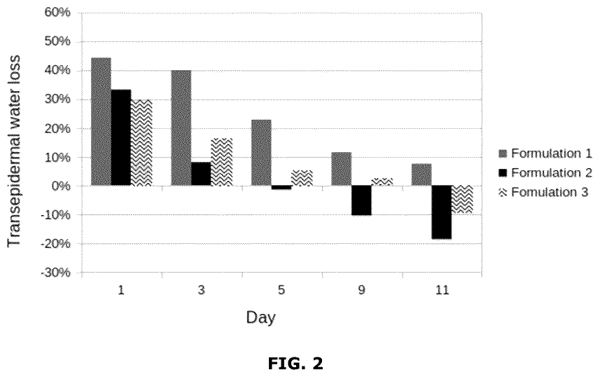

TEWL measurements were collected every second over a period of 40 seconds. The TEWL values reported are an average of 20 measurements, taken in the period 21 to 40 seconds, excluding the first 20 seconds to give the probe time to acclimatise to the skin temperature. The results are shown in FIG. 2.

### Example 5: Skin Irritation and Sensitisation

A total of 44 male or female subjects, ranging in age from 19 to 70 years, with a self-perceived sensitive skin but otherwise a history of normal skin conditions, were selected for study participation. Subjects were asked to avoid excessive sun exposure, refrain from getting patches wet or washing test areas with any soap or other products, and not to introduce any new cosmetic or toiletry product during the course of the study.

For the induction phase, test sites were cleansed with 70% isopropyl alcohol and allowed to dry. Approximately 0.2 g of the test material (Formulation 2) was then applied to the fabric portion of a patch to the upper back, between the scapulae and the waist, to either side of the spinal midline, and then subsequently applied three times a week on Monday, Wednesday and Friday, for a total of 9 applications. The patch consisted of a semi-occlusive strip of breathable tape with a centre portion of 2 cm fabric. Subjects removed the patch after 24 hours after exposure. An evaluation of each site was made just prior to the application of the next patch for signs of dermal reactions. Evaluations were scored as:

| Score | Description |
|---|---|
| 0 | No visible skin reactions |
| ± | Barely perceptible erythema |
| 1+ | Mild erythema |
| 2+ | Well defined erythema |
| 3+ | Severe erythema and edema |
| 4+ | Erythema and edema with vesiculation |

Approximately 10 to 21 days after the final induction phase visit, a challenge patch was applied to a virgin site on the lower back, following the same procedure described for the induction phase. After 24 hours, the patch was removed and the site then evaluated and scored for sensitisation. All subjects had scores of ‘0’ showing no dermal reactions throughout the course of the entire study.

### Example 6: Sample Preparation for Trial 2

### Heat Treatment

Heat treatment was performed using an electric plate heater with gentle stirring providing by an overhead stirrer fitted with a blade (40 length by 7 mm high) at a speed of 100 rpm. A 512 g quantity of mamaku extract was placed in a metal beaker (90 mm diameter) and heated from room temperature to a target temperature of 70° C. The temperature was controlled with a thermostatic temperature controller (Kegland MKII) and the temperature was recorded using a Raspberry pi single-board computer (model 4 B) connected to a waterproof DS180B20 digital temperature probe.

### Ultrasound Treatment

Ultrasound treatment was performed using a Hielscher UIP1000hd (Hielscher systems GmbH, Germany) device that delivers ultrasound at 20 kHz at 1 kW with an amplitude of 25 μm. The system was fitted with a B2-1.8 booster horn to increase the amplitude by a ratio of 1:1.8 to a 40 mm sonotrode (BS4d40) and the amplitude controller was set to 100% output. Ultrasound was performed on a 500 g batch of mamaku extract placed in a 2 L plastic jug with a base diameter of 100 mm. Ultrasound was applied in pulses of 10 s, with rests of 10 s between consecutive pulses.

### Agitation

Mechanical agitation was provided by a food blender (NutriBullet 1000). A 500 g quantity of mamaku extract was processed at the high-speed setting for 5 s at room temperature. The blender has a top speed of 20,300 rpm and a 1 kW rating. There are 4 cutting blades, angled at the base of the blender each with a length of approximately 30 mm.

### Mock Cosmetic Products

Mock cosmetic products were prepared that contained cosmetic grade 200 mesh xanthan gum (1.9% w/w), glycerol (3.0% w/w), untreated, heat treated, ultrasound treated or agitation treated mamaku extracts (50%) and water (45.1%). A base mixture was first prepared by making a slurry consisting of the xanthan gum and glycerol components, and then slowly adding water, with stirring, until all three components were mixed in. This was then left overnight to allow the gum to fully hydrate. Mamaku extract was then added to the base mixture and mixed gently until a homogenous gel was formed.

### Example 7: Spreadability

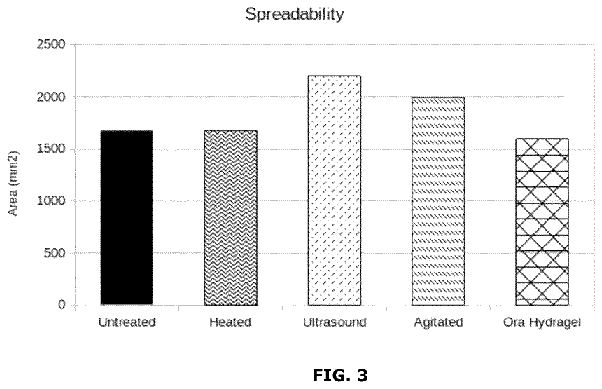

The spreadability of the mock cosmetic products of Example 6 was tested by spreading a 1 g quantity of a sample of each product between two glass plates with a 50 g mass on the upper plate. After 60 s, the diameter of the spread was measured. All mock cosmetic products had spreadability that was similar to or greater than the commercial mamaku gel product Ora Hydrogel. The results are shown in FIG. 3.

### Example 8: Viscosity

Rheological measurements were performed using an Anton Parr MCR 302 rheometer, with a cone and plate geometry (CP50-1) with a 0.1 mm gap. Care was taken to load an amount of mamaku extract onto the plate that was just enough to completely fill the gap all around when the cone was lowered. Standard measurements were performed at 20° C., which was controlled by a water bath and a Peltier system, over a frequency range from 0.1 to 100 Hz in rotational mode. The results are shown in FIG. 4.

### Example 9: Trial 2 Procedure

The study comprised a total of 17 healthy participants, with an average age of 45 and an age range of 22 to 65 (median of 49). Participants were instructed not to use any cosmetic products, aside from their standard soap/body wash, on their forearms for the duration of, and including two weeks prior to, the study. Each participant was supplied with a pack to take home containing the three formulations (untreated, heat treated and ultrasound treated) and information, including a diagram indicating how much of the formulations to use and on which test site to reproducibly apply the different treatments. The participants were also provided with a diary to fill in what time, in the morning and evening, they applied the formulations, and whether they showered/bathed in the morning or evening, or both. The trial was conducted over 10 days and the formulations were applied twice daily, morning and evening, to both arms. This commenced with the first application after the baseline measurement on Day 1 and the last application was on the morning of Day 10. Baseline readings were taken on Day 1 and readings were also taken on Days 3, 5 and 10.

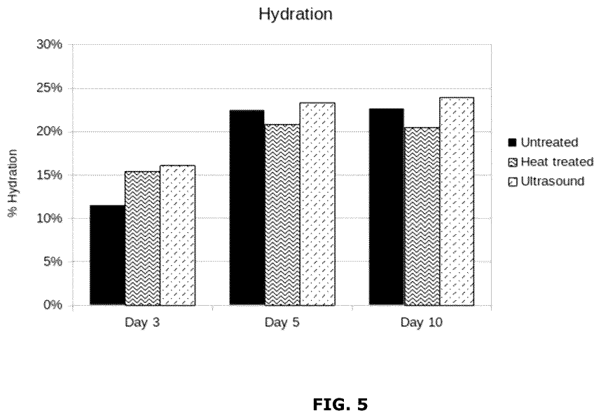

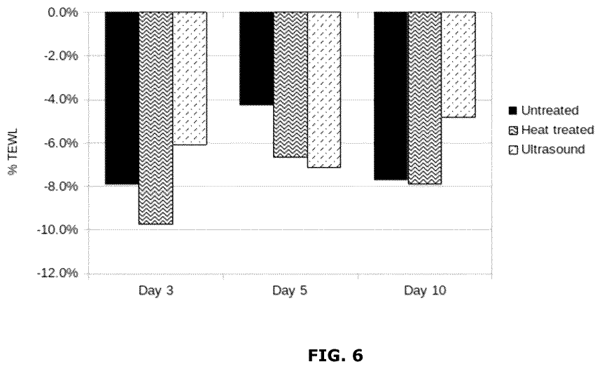

Hydration was measured with a corneometer (CM825) and TEWL was measured with a Tewameter (TM 300) from Courage and Khazaka electronic GmbH, Köln, Germany. All measurements were performed following a 15-minute acclimatisation period oat 20° C. and 50% relative humidity. The hydration measurements are shown in FIG. 5. The TEWL measurements are shown in FIG. 6.

### Example 10: Trial 3 Procedure

A total of 67 participants (47 females, 70%) participated in this trial. The age ranged from 2 weeks to 63 years (mean 21.7 yrs). Participants were asked to provide demographic information (age, sex) and details of their underlying skin problem, including previous treatments. Diagnosis included atopic eczema (45%), hand dermatitis (15%), other eczemas (12%), acne (9%), and psoriasis (6%) (see Table 1). They were asked to take cell-phone images of the affected skin before, and two weeks after, twice-daily application of Formulation 2 made according to Example 1 containing 0.85% mamaku extract solids. They were also asked to provide a free text commentary on the effects on their skin, the tolerability of the product, and their general experience. These narratives and images were collated, de-identified and sent to an independent dermatologist for comment. The dermatologist reviewed the supplied narrative and clinical images, determined the underlying dermatological diagnoses, and categorised the response as: clear skin, significant improvement, moderate improvement, slight improvement, and no improvement. The results are shown in Table 1.

TABLE 1
Trial results

| | | | Age | | | | | |
|---|---|---|---|---|---|---|---|---|
| | | | (years) | Improvement | | | | |
| Diagnosis | n | Male/Female | (range) | Clear | Significant | Moderate | Slight | None |
| Atopic | 30 | 14/16 | 9.0 | 1 | 9 | 18 | 2 | 0 |
| eczema | | | (0.75-33) | | | | | |
| Hand | 10 | 3/7 | 42.0 | 0 | 2 | 6 | 0 | 2 |
| dermatitis | | | (16-55) | | | | | |
| Eczema - | 8 | 1/7 | 34 | 1 | 1 | 5 | 1 | 0 |
| other | | | (10-49) | | | | | |
| Acne | 6 | 0/6 | 29.5 | 0 | 1 | 4 | 1 | 0 |
| | | | (11-39) | | | | | |
| Psoriasis | 4 | 1/3 | 34 | 0 | 1 | 2 | 1 | 0 |
| | | | (1-55) | | | | | |
| Rosacea | 2 | 0/2 | 37.0 | 0 | 1 | 1 | 0 | 0 |
| Other | 7 | 1/6 | 29.0 | 0 | 3 | 3 | 1 | 0 |
| | | | (1/12-63) | | | | | |
| Total (%) | 67 | 20/47 | 21.7 | 2 | 18 | 39 | 6 | 2 |
| | | (30%/70%) | (1/12-63) | (3%) | (27%) | (58%) | (9%) | (3%) |

1. An aqueous extract of the black tree fern Cyathea medullaris wherein the extract is prepared by:
(i) cutting fronds of Cyathea medullaris to give plant material having an average portion size of 0.1 to 10 cm;
(ii) heating a mixture of the plant material of step (i) and water at a temperature of 45 to 60° C. with laminar mixing or transitional flow mixing; and
(iii) removing solid material from the mixture to give the aqueous extract.

2. The extract as claimed in claim 1, wherein the amount of plant material in water in step (ii) is 20-30% by weight.

3. The extract as claimed in claim 1, wherein the plant material is heated in step (ii) for 30 minutes to 2 hours.

4. The extract as claimed in claim 1, wherein the average portion size of the plant material is 0.2 to 5 cm.

5. The extract as claimed in claim 1, wherein the temperature of the water in step (ii) is 50-55° C.

6. The extract as claimed in claim 1, wherein removing the solid material in step (iii) is by filtration under laminar conditions.

7. The extract as claimed in claim 1, wherein the extract comprises glucuronomannan polysaccharides in the amount of 0.05 to 0.1% by weight of the extract.

8. The extract as claimed in claim 7, wherein the extract additionally comprises glycerin, xanthan gum, or a combination thereof.

9. The extract as claimed in claim 1, wherein the plant material comprises fronds of the Cyathea medullaris plant.

10. A topical composition for use in treating eczema comprising the extract of claim 1.

11. A composition as claimed in claim 10, further comprising one or more preservatives.

12. A composition as claimed in claim 11, wherein the one or more preservatives are selected from dehydroacetic acid and benzyl alcohol.

13. A composition as claimed in claim 11, further comprising xanthan gum, glycerin, or a combination thereof.

14. A method of treating eczema comprising administering to a subject an aqueous extract of the black tree fern Cyathea medullaris, wherein the extract is prepared by:
(i) cutting fronds of Cyathea medullaris to give plant material having an average portion size of 0.1 to 10 cm,
(ii) heating a mixture of the plant material of step (i) and water at a temperature of 45 to 60° C. with laminar mixing or transitional flow mixing; and
(iii) removing solid material from the mixture to give the aqueous extract.

15. A process for preparing an aqueous extract of the black tree fern Cyathea medullaris comprising:
(i) cutting fronds of Cyathea medullaris to give plant material having an average portion size of 0.1 to 10 cm;
(ii) heating a mixture of the plant material of step (i) and water at a temperature of 45 to 60° C. with laminar mixing or transitional flow mixing; and
(iii) removing solid material from the mixture to give the aqueous extract.

16. The extract as claimed in claim 2, wherein the plant material is heated in step (ii) for 30 minutes to 2 hours.

17. The extract as claimed in claim 16, wherein the average portion size of the plant material is 0.2 to 5 cm.

18. The extract as claimed in claim 17, wherein the temperature of the water in step (ii) is 50-55° C.

19. The extract as claimed in claim 18, wherein the extract comprises glucuronomannan polysaccharides in the amount of 0.05 to 0.1% by weight of the extract.

20. The extract as claimed in claim 19, wherein the plant material comprises fronds of the Cyathea medullaris plant.
